(19) United States
(12) Patent Application Publication
(10) Pub. No.: US 2007/0293492 A1
(43) Pub. Date: Dec. 20, 2007

(54) Quinoline Tachykinin Receptor Antagonists

(76) Inventors: Robert J. DeVita, Westfield, NJ (US); Jinlong Jiang, Scotch Plains, NJ (US); Sander G. Mills, Scotch Plains, NJ (US)

Correspondence Address: MERCK AND CO., INC, P O BOX 2000, RAHWAY, NJ, 07065-0907, US

(21) Appl. No.: 11/791,241

(22) PCT Filed: Nov. 29, 2005
(86) PCT No.: PCT/US05/43130

§ 371 Date: May 21, 2007

Related U.S. Application Data
(60) Provisional application No. 60/632,861, filed on Dec. 3, 2004.

(51) Int. Cl. A61K 31/47; A61K 31/535; A61P 25/00; C07D 215/38; C07D 413/02

(52) U.S. Cl. 514/235.2; 514/311; 514/313; 544/128; 546/159; 546/171

(57) ABSTRACT

The present invention is directed to certain quinoline compounds which are useful as neurokinin-1 (NK-1) receptor antagonists, and inhibitors of tachykinin and in particular substance P. The invention is also concerned with pharmaceutical formulations comprising these compounds as active ingredients and the use of the compounds and their formulations in the treatment of certain disorders, including emesis, urinary incontinence, depression, and anxiety.

### BACKGROUND OF THE INVENTION

Substance P is a naturally occurring undecapeptide belonging to the tachykinin family of peptides, the latter being so-named because of their prompt contractile action on extravascular smooth muscle tissue. The tachykinins are distinguished by a conserved carboxyl-terminal sequence. In addition to substance P, the known mammalian tachykinins include neurokinin A and neurokinin B. The current nomenclature designates the receptors for substance P, neurokinin A, and neurokinin B as neurokinin-1 (NK-1), neurokinin-2 (NK-2), and neurokinin-3 (NK-3), respectively. Tachykinin, and in particular substance P, antagonists are useful in the treatment of clinical conditions which are characterized by the presence of an excess of tachykinin, in particular substance P, activity, including disorders of the central nervous system, nociception and pain, gastrointestinal disorders, disorders of bladder function and respiratory diseases. Attempts have been made to provide antagonists for the receptors of substance P and other tachykinin peptides in order to more effectively treat the various disorders and diseases mentioned above.

### SUMMARY OF THE INVENTION

The present invention is directed to certain quinoline compounds which are useful as neurokinin-1 (NK-1) receptor antagonists, and inhibitors of tachykinin and in particular substance P. The invention is also concerned with pharmaceutical formulations comprising these compounds as active ingredients and the use of the compounds and their formulations in the treatment of certain disorders, including emesis, urinary incontinence, depression, and anxiety.

### DETAILED DESCRIPTION OF THE INVENTION

The present invention is directed to compounds of the formula I: 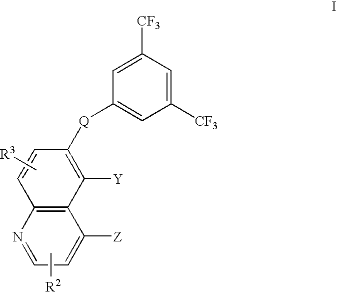
and N-oxides thereof,
wherein:
Q is selected from the group consisting of:
(1) —O—CH₂—,
(2) —O—CH(CH₃)—,
(3) —O—CH(CH₂OH)—,
(4) —N(R⁵)—C(R⁶R⁷)—,
   wherein R⁵, R⁶ and R⁷ are independently selected from:
   (a) hydrogen, and
   (b) —CH₃,
(5) —N(R⁵)—,
(6) —N(R⁵)—CO—C(R⁶R⁷)—, and
(7) —N(R⁵)—CH₂—C(R⁶R⁷)—;
   Y and Z are selected from hydrogen and phenyl, wherein one of Y and Z is hydrogen and the other of Y and Z is phenyl, and wherein the phenyl is substituted with R¹², R¹³ and R¹⁴, where R¹², R¹³ and R¹⁴ are independently selected from:
(1) hydrogen,
(2) halo, and
(3) C₁₋₆ alkyl;
   R² and R³ are independently selected from the group consisting of:
(1) hydrogen,
(2) C₁₋₆ alkyl, which is unsubstituted or substituted with one or more of the substituents selected from:
   (a) hydroxy,
   (b) oxo,
   (c) C₁₋₆ alkoxy,
   (d) phenyl-C₁₋₃ alkoxy,
   (e) phenyl,
   (f) halo,
   (g) —NR⁹R¹⁰, wherein R⁹ and R¹⁰ are independently selected from:
      (I) hydrogen,
      (II) C₁₋₆ alkyl,
      (III) phenyl,
      (IV) (C₁₋₆ alkyl)-phenyl,
      (V) (C₁₋₆ alkyl)-hydroxy, and
      (VI) (C₁₋₆ alkyl)-(C₁₋₄ alkoxy),
      or where —NR⁹R¹⁰ forms a morpholine, piperidine or quinuclidine ring
   (h) —NR⁹—COR¹¹, wherein R¹¹ is independently selected from:
      (I) hydrogen,
      (II) C₁₋₆ alkyl,
      (III) phenyl,
      (IV) (C₁₋₆ alkyl)-phenyl,
      (V) (C₁₋₆ alkyl)-hydroxy, and
      (VI) (C₁₋₆ alkyl)-(C₁₋₄ alkoxy),
   (j) —NR⁹—CO₂R¹¹,
   (k) —CO—NR⁹R¹⁰,
   (l) —COR¹¹,
   (m) —CO₂R¹¹,
(3) hydroxy,
(4) C₁₋₆alkoxy,
(5) oxo,
(6) halo,
(7) —CN,
(8) —CF₃,
(9) —NR⁹R¹⁰,
(10) —NR⁹—COR¹¹,
(11) —NR⁹—CO₂R¹¹,
(12) —CO—NR⁹—COR¹¹,
(13) —COR¹¹,
(14) —O—(CO)R¹¹,
(15) —CO₂R¹¹,
(16) -imidazolyl, and
(17) -triazolyl;
   and pharmaceutically acceptable salts thereof and individual enantiomers and diastereomers thereof.

An embodiment of the present invention includes compounds of the formula Ia: 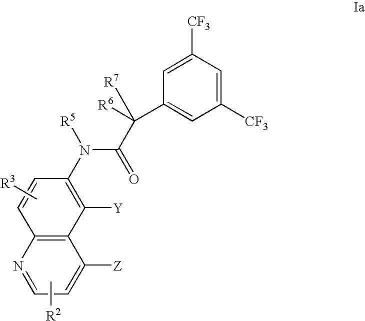
wherein R², R³, R⁵, R⁶ and R⁷ are defined herein;
and pharmaceutically acceptable salts thereof and individual enantiomers and diastereomers thereof.

An embodiment of the present invention includes compounds of the formula Ib: 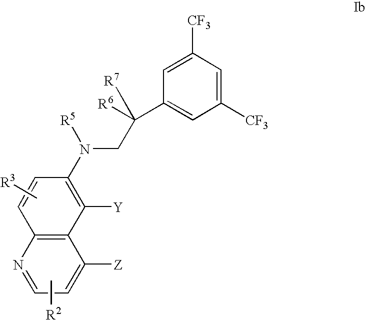
wherein R², R³, R⁵, R⁶ and R⁷ are defined herein;
and pharmaceutically acceptable salts thereof and individual enantiomers and diastereomers thereof.

An embodiment of the present invention includes compounds of the formula Ic: 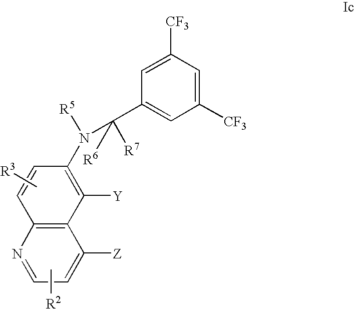
wherein R², R³, R⁵, R⁶ and R⁷ are defined herein;
and pharmaceutically acceptable salts thereof and individual enantiomers and diastereomers thereof.

An embodiment of the present invention includes compounds of the formula Id: 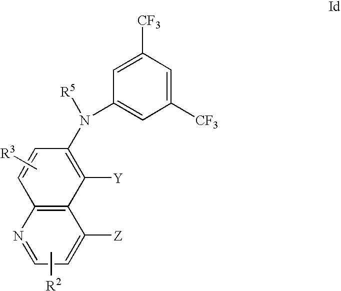
wherein R², R³, R⁵, R⁶ and R⁷ are defined herein;
and pharmaceutically acceptable salts thereof and individual enantiomers and diastereomers thereof.

An embodiment of the present invention includes compounds of the formula Ie: 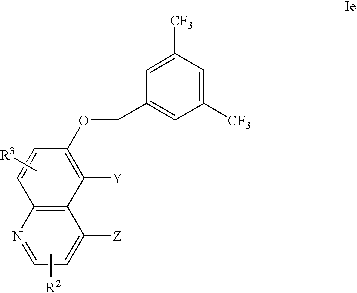
wherein R² and R³ are defined herein;
and pharmaceutically acceptable salts thereof and individual enantiomers and diastereomers thereof.

An embodiment of the present invention includes compounds wherein Y is phenyl and Z is hydrogen.

An embodiment of the present invention includes compounds wherein Y is hydrogen and Z is phenyl.

An embodiment of the present invention includes compounds wherein Y is 2-methyl-phenyl and Z is hydrogen.

An embodiment of the present invention includes compounds wherein Y is hydrogen and Z is 2-methyl-phenyl.

An embodiment of the present invention includes compounds wherein R² is selected from the group consisting of:
(1) hydrogen,
(2) morpholinyl,
(3) quinuclidinyl,
(4) C₁₋₆ alkyl, which is unsubstituted or substituted with one or more of the substituents selected from:
   (a) morpholinyl,
   (b) —CO₂(C₁₋₆ alkyl), and
   (c) —CO₂H,
(5) hydroxy,
(6) —CO₂H, and
(7) —CN.

Within this embodiment, the present invention includes compounds wherein R² is hydrogen.

Also within this embodiment, the present invention includes compounds wherein R² is morpholinyl.

An embodiment of the present invention includes compounds wherein R² is hydrogen.

An embodiment of the present invention includes compounds wherein R³ is hydrogen.

An embodiment of the present invention includes compounds wherein the compound is present as an N-oxide on the quinuclidinyl ring.

Specific embodiments of the present invention include a compound which is selected from the group consisting of the subject compounds of the Examples herein and pharmaceutically acceptable salts thereof and individual enantiomers and diastereomers thereof.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds. Formula I shows the structure of the class of compounds without preferred stereochemistry. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art. Alternatively, any enantiomer of a compound may be obtained by stereoselective synthesis using optically pure starting materials or reagents of known configuration by methods well known in the art.

There are several acceptable methods of naming the compounds discussed herein.

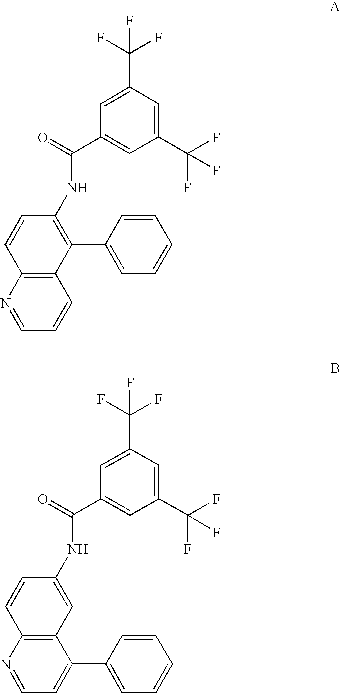

For example, the above compound A can be named as “N-(5-phenylquinolin-6-yl)-3,5-bis(trifluoromethyl)benzamide.” The core structure A may be generally referred to as a 5-phenylquinoline. Similarly compound B can be named as “N-(4-phenylquinolin-6-yl)-3,5-bis(trifluoromethyl)-benzamide”. The core structure B may be generally referred to as a 4-phenylquinoline.

As appreciated by those of skill in the art, halo or halogen as used herein are intended to include fluoro, chloro, bromo and iodo. Similarly, C₁₋₆, as in C₁₋₆alkyl is defined to identify the group as having 1, 2, 3, 4, 5 or 6 carbons in a linear or branched arrangement, such that C₁₋₆alkyl specifically includes methyl, ethyl, n-propyl, iso-propyl, n-butyl, iso-butyl, tert-butyl, pentyl and hexyl. A group which is designated as being independently substituted with substituents may be independently substituted with multiple numbers of such substituents.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are benzenesulfonic, citric, hydrobromic, hydrochloric, maleic, fumaric, succinic and tartaric acids. It will be understood that, as used herein, references to the compounds of the present invention are meant to also include the pharmaceutically acceptable salts.

Exemplifying the invention is the use of the compounds disclosed in the Examples and herein. Specific compounds within the present invention include a compound which selected from the group consisting of the compounds disclosed in the following Examples and pharmaceutically acceptable salts thereof and individual diastereomers thereof.

General procedures for the preparation of compounds are described in the following schemes. Substituted or unsubstituted quinolines A may be nitrated under conditions known to those skilled in the art to provide 6-nitroquinoline intermediates B. The nitro group of these compounds may be reduced under a variety of conditions to provide 6-aminoquinoline compounds C. The amino compounds may be treated with bromine to provide 6-amino-5-bromo derivatives which may be protected with protecting groups such as BOC- or CBZ-protecting groups known to those skilled in the art to provide intermediates of general structure D. The bromine may be reacted in a an aryl cross-coupling reaction with such as aryl metallic reagents such as aryl stannane or boronic acid with a variety of metal catalyst such as Pd to provide 5-aryl quinolone intermediates of general structure E. The nitrogen protecting groups made removed under a variety of conditions know to those skilled in the art to provide 6-aminoquinoline intermediates which may be functionalized on nitrogen by reactions such as carboxamide eformation, amination reactions, urea formation or carbamate formation to provide compounds of the present invention of general structure I (wherein R¹ is the Q-((3,5-bis-trifluoromethyl)phenyl) group).

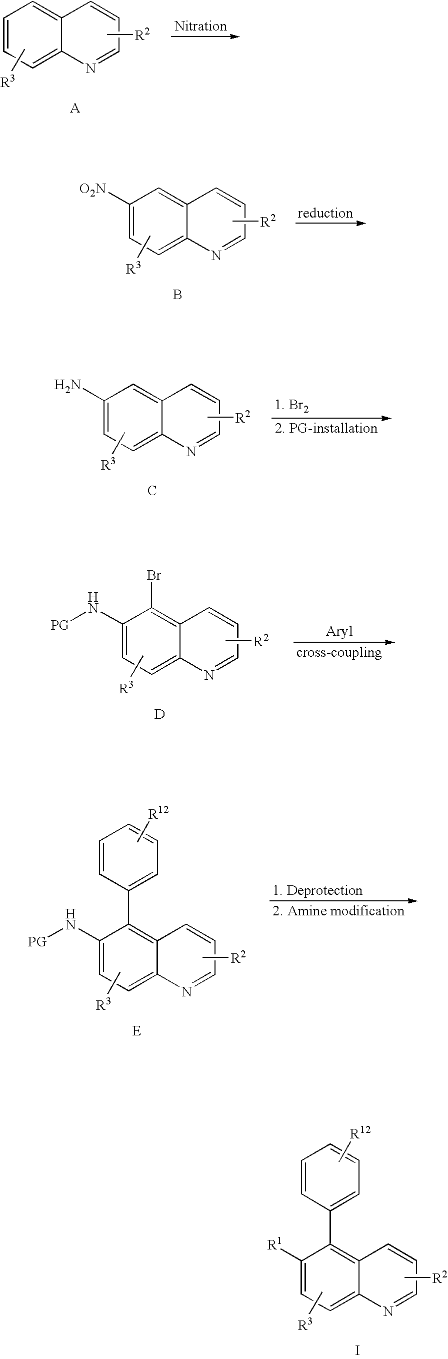

Chemistry for further modifications at the 6-position is described in the next scheme. The above 6-amino intermediate C may be converted to the diazonium salt by a variety of conditions such an HNO₂. The crude diazonium salt may be decomposed in the presence of H₂SO₄ and ice water to provide the 6-hydroxyquinoline intermediate F. The 6-hydroxyl group may be functionalized by a variety of reaction such as etherification, esterification, carbamate formation to provide 6-oxy compounds of the present invention of the general structure I.

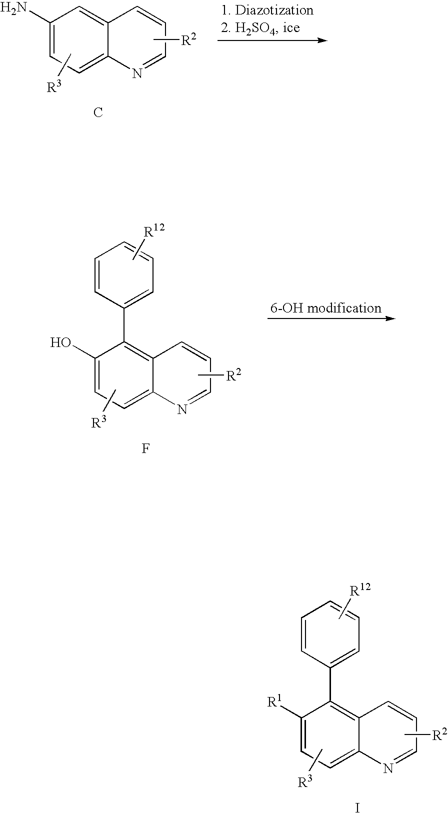

Similar reaction sequences may be utilized to prepare compounds of general structure II. 2,4-dihyroxyquinoline derivatives G may be nitrated with nitric acid to provide 2,4-dihydroxy-6-nitroquinoline intermediates H. The 2,4-hydroxyl grouped made be converted to chloro groups by the treatment of POCl₃ or other reagents known to those skilled in the art to provide the 2,4-dichloro-6-nitroquinoline compounds I. The 2-chloro group may be selectively functionalized under a variety of reactions such as amine substitution, organometallic cross coupling or other reaction of 2-heteroaryl chlorides to provide 2-substituted intermediates of general structure J. The 4-chloro group may be reacted with a variety of aryl metal reagents such as aryl stannane or aryl boronic acids with metal catalysis such Pd-catalysts to provide intermediates K. The 6-nitro group may be reduced to the 6-amino intermediate L by hydrogenation or chemical reduction. The 6-amino group may be functionalized a described above to provide compounds of the present invention, of generalized structures II.

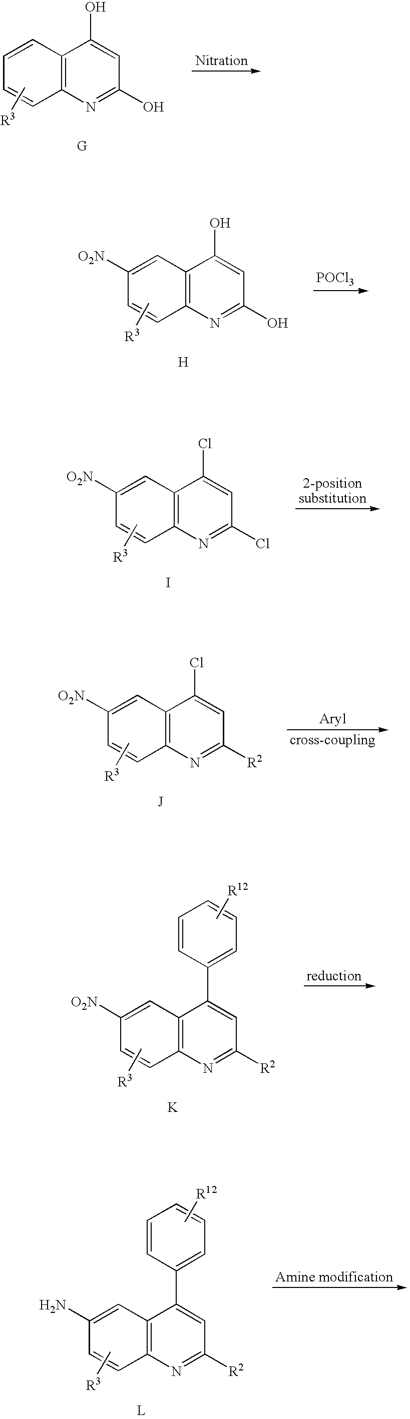

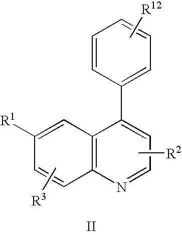

Similar to the procedures described above, intermediates of general structure L may be diazotized and decomposed to provide 6-hydroxy-4-phenylquinoline intermediates M. The 6-hydroxyl groups of these compounds may be further functionalized to provide ethers, ester and carbamates by procedures known to those skilled in the art to provide 6-oxy analogs of the present invention of generalized structure II.

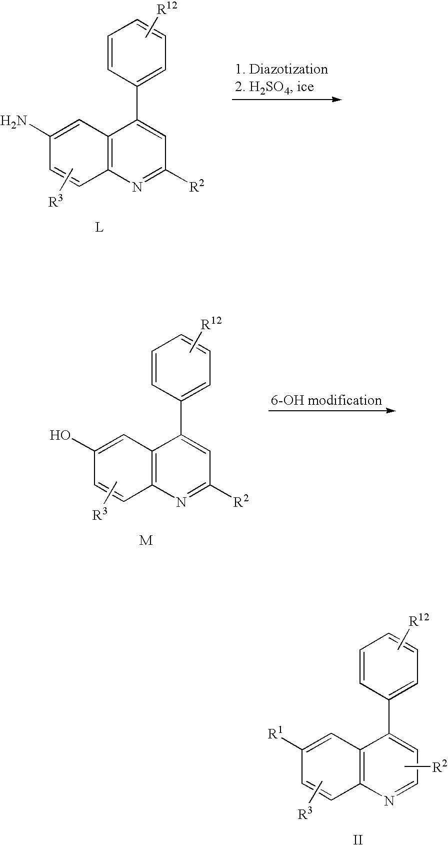

These generalized compounds I and II may also serve as intermediates and may be further substituted or functionalized by reactions know to those skilled in the art and outlined in detail in the experimentals contained herein.

The compounds of the present invention are useful in the prevention and treatment of a wide variety of clinical conditions which are characterized by the presence of an excess of tachykinin, in particular substance P, activity. Thus, for example, an excess of tachykinin, and in particular substance P, activity is implicated in a variety of disorders of the central nervous system. Such disorders include mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder; anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalised anxiety disorders; schizophrenia and other psychotic disorders, for example, schizophreniform disorders, schizoaffective disorders, delusional disorders, brief psychotic disorders, shared psychotic disorders and psychotic disorders with delusions or hallucinations; delirium, dementia, and amnestic and other cognitive or neurodegenerative disorders, such as Alzheimer's disease, senile dementia, dementia of the Alzheimer's type, vascular dementia, and other dementias, for example, due to HIV disease, head trauma, Parkinson's disease, Huntington's disease, Pick's disease, Creutzfeldt-Jakob disease, or due to multiple etiologies; Parkinson's disease and other extra-pyramidal movement disorders such as medication-induced movement disorders, for example, neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor; substance-related disorders arising from the use of alcohol, amphetamines (or amphetamine-like substances), caffeine, cannabis, cocaine, hallucinogens, inhalants and aerosol propellants, nicotine, opioids, phenylglycidine derivatives, sedatives, hypnotics, and anxiolytics, which substance-related disorders include dependence and abuse, intoxication, withdrawal, intoxication delirium, withdrawal delirium, persisting dementia, psychotic disorders, mood disorders, anxiety disorders, sexual dysfunction and sleep disorders; epilepsy; Down's syndrome; demyelinating diseases such as MS and ALS and other neuropathological disorders such as peripheral neuropathy, for example diabetic and chemotherapy-induced neuropathy, and postherpetic neuralgia, trigeminal neuralgia, segmental or intercostal neuralgia and other neuralgias; and cerebral vascular disorders due to acute or chronic cerebrovascular damage such as cerebral infarction, subarachnoid hemorrhage or cerebral oedema.

Tachykinin, and in particular substance P, activity is also involved in nociception and pain. The compounds of the present invention will therefore be of use in the prevention or treatment of diseases and conditions in which pain predominates, including soft tissue and peripheral damage, such as acute trauma, osteoarthritis, rheumatoid arthritis, musculo-skeletal pain, particularly after trauma, spinal pain, myofascial pain syndromes, headache, episiotomy pain, and burns; deep and visceral pain, such as heart pain, muscle pain, eye pain, orofacial pain, for example, odontalgia, abdominal pain, gynecological pain, for example, dysmenorrhea, and labour pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; pain associated with carcinoma, often referred to as cancer pain; central nervous system pain, such as pain due to spinal cord or brain stem damage; low back pain; sciatica; ankylosing spondylitis, gout; and scar pain.

Tachykinin, and in particular substance P, antagonists may also be of use in the treatment of respiratory diseases, particularly those associated with excess mucus secretion, such as chronic obstructive airways disease, bronchopneumonia, chronic bronchitis, cystic fibrosis and asthma, adult respiratory distress syndrome, and bronchospasm; inflammatory diseases such as inflammatory bowel disease, psoriasis, fibrositis, osteoarthritis, rheumatoid arthritis, pruritis and sunburn; allergies such as eczema and rhinitis; hypersensitivity disorders such as poison ivy; ophthalmic diseases such as conjunctivitis, vernal conjunctivitis, and the like; ophthalmic conditions associated with cell proliferation such as proliferative vitreoretinopathy; cutaneous diseases such as contact dermatitis, atopic dermatitis, urticaria, and other eczematoid dermatitis. Tachykinin, and in particular substance P, antagonists may cell carcinomas such as small cell lung cancer.

Tachykinin, and in particular substance P, antagonists may also be of use in the treatment of gastrointestinal (GI) disorders, including inflammatory disorders and diseases of the GI tract such as gastritis, gastroduodenal ulcers, gastric carcinomas, gastric lymphomas, disorders associated with the neuronal control of viscera, ulcerative colitis, Crohn's disease, irritable bowel syndrome and emesis, including acute, delayed or anticipatory emesis such as emesis induced by chemotherapy, radiation, toxins, viral or bacterial infections, pregnancy, vestibular disorders, for example, motion sickness, vertigo, dizziness and Meniere's disease, surgery, migraine, variations in intercranial pressure, gastro-oesophageal reflux disease, acid indigestion, over indulgence in food or drink, acid stomach, waterbrash or regurgitation, heartburn, for example, episodic, nocturnal or meal-induced heartburn, and dyspepsia.

Tachykinin, and in particular substance P, antagonists may also be of use in the treatment of a variety of other conditions including stress related somatic disorders; reflex sympathetic dystrophy such as shoulder/hand syndrome; adverse immunological reactions such as rejection of transplanted tissues and disorders related to immune enhancement or suppression such as systemic lupus erythematosus; plasma extravasation resulting from cytokine chemotherapy, disorders of bladder function such as cystitis, bladder detrusor hyper-reflexia, frequent urination and urinary incontinence, including the prevention or treatment of overactive bladder with symptoms of urge urinary incontinence, urgency, and frequency; fibrosing and collagen diseases such as scleroderma and eosinophilic fascioliasis; disorders of blood flow caused by vasodilation and vasospastic diseases such as angina, vascular headache, migraine and Reynaud's disease; and pain or nociception attributable to or associated with any of the foregoing conditions, especially the transmission of pain in migraine. The compounds of the present invention are also of value in the treatment of a combination of the above conditions, in particular in the treatment of combined post-operative pain and post-operative nausea and vomiting.

The compounds of the present invention are particularly useful in the prevention or treatment of emesis, including acute, delayed or anticipatory emesis, such as emesis induced by chemotherapy, radiation, toxins, pregnancy, vestibular disorders, motion, surgery, migraine, and variations in intercranial pressure. For example, the compounds of the present invention are of use optionally in combination with other antiemetic agents for the prevention of acute and delayed nausea and vomiting associated with initial and repeat courses of moderate or highly emetogenic cancer chemotherapy, including high-dose cisplatin. Most especially, the compounds of the present invention are of use in the treatment of emesis induced by antineoplastic (cytotoxic) agents, including those routinely used in cancer chemotherapy, and emesis induced by other pharmacological agents, for example, rolipram. Examples of such chemotherapeutic agents include alkylating agents, for example, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; and cytotoxic antibiotics. Particular examples of chemotherapeutic agents are described, for instance, by D. J. Stewart in Nausea and Vomiting: Recent Research and Clinical Advances, Eds. J. Kucharczyk et al, CRC Press Inc., Boca Raton, Fla., USA (1991) pages 177-203, especially page 188. Commonly used chemotherapeutic agents include cisplatin, dacarbazine (DTIC), dactinomycin, mechlorethamine, streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin and chlorambucil [R. J. Gralla et al in Cancer Treatment Reports (1984) 68(1), 163-172]. A further aspect of the present invention comprises the use of a compound of the present invention for achieving a chronobiologic (circadian rhythm phase-shifting) effect and alleviating circadian rhythm disorders in a mammal. The present invention is further directed to the use of a compound of the present invention for blocking the phase-shifting effects of light in a mammal.

The present invention is further directed to the use of a compound of the present invention or a pharmaceutically acceptable salt thereof, for enhancing or improving sleep quality as well as preventing and treating sleep disorders and sleep disturbances in a mammal. In particular, the present invention provides a method for enhancing or improving sleep quality by increasing sleep efficiency and augmenting sleep maintenance. In addition, the present invention provides a method for preventing and treating sleep disorders and sleep disturbances in a mammal which comprising the administration of a compound of the present invention or a pharmaceutically acceptable salt thereof. The present invention is useful for the treatment of sleep disorders, including Disorders of Initiating and Maintaining Sleep (insomnias) (“DIMS”) which can arise from psychophysiological causes, as a consequence of psychiatric disorders (particularly related to anxiety), from drugs and alcohol use and abuse (particularly during withdrawal stages), childhood onset DIMS, nocturnal myoclonus, fibromyalgia, muscle pain, sleep apnea and restless legs and non specific REM disturbances as seen in ageing.

The particularly preferred embodiments of the instant invention are the treatment of emesis, urinary incontinence, depression or anxiety by administration of the compounds of the present invention to a subject (human or companion animal) in need of such treatment.

The present invention is directed to a method for the manufacture of a medicament for antagonizing the effect of substance P at its receptor site or for the blockade of neurokinin-1 receptors in a mammal comprising combining a compound of the present invention with a pharmaceutical carrier or diluent. The present invention is further directed to a method for the manufacture of a medicament for the treatment of a physiological disorder associated with an excess of tachykinins in a mammal comprising combining a compound of the present invention with a pharmaceutical carrier or diluent.

The present invention also provides a method for the treatment or prevention of physiological disorders associated with an excess of tachykinins, especially substance P, which method comprises administration to a patient in need thereof of a tachykinin reducing amount of a compound of the present invention or a composition comprising a compound of the present invention. As used herein, the term “treatment” or “to treat” refers to the administration of the compounds of the present invention to reduce, ameliorate, or eliminate either the symptoms or underlying cause of the noted disease conditions, in a subject (human or animal) that suffers from that condition or displays clinical indicators thereof. The term “prevention” or “to prevent” refers to the administration of the compounds of the present invention to reduce, ameliorate, or eliminate the risk or likelihood of occurrence of the noted disease conditions, in a subject (human or animal) susceptible or predisposed to that condition.

The compounds of this invention are useful for antagonizing tachykinins, in particular substance P in the treatment of gastrointestinal disorders, central nervous system disorders, inflammatory diseases, pain or migraine and asthma in a mammal in need of such treatment. This activity can be demonstrated by the following assays.

Receptor Expression in COS: To express the cloned human neurokinin-1 receptor (NK1R) transiently in COS, the cDNA for the human NK1R was cloned into the expression vector pCDM9 which was derived from pCDM8 (INVITROGEN) by inserting the ampicillin resistance gene (nucleotide 1973 to 2964 from BLUESCRIPT SK+) into the Sac II site. Transfection of 20 ug of the plasmid DNA into 10 million COS cells was achieved by electroporation in 800 ul of transfection buffer (135 mM NaCl, 1.2 mM CaCl₂, 1.2 mM MgCl₂, 2.4 mM K₂HPO₄, 0.6 mM KH₂PO₄, 10 mM glucose, 10 mM HEPES pH 7.4) at 260 V and 950 uF using the IBI GENEZAPPER (IBI, New Haven, Conn.). The cells were incubated in 10% fetal calf serum, 2 mM glutamine, 100 U/ml penicillin-streptomycin, and 90% DMEM media (GIBCO, Grand Island, N.Y.) in 5% CO₂ at 37° C. for three days before the assay.

Stable Expression in CHO: To establish a stable cell line expressing the cloned human NK1R, the cDNA was subcloned into the vector pRcCMV (INVITROGEN). Transfection of 20 ug of the plasmid DNA into CHO cells was achieved by electroporation in 800 ul of transfection buffer supplemented with 0.625 mg/ml Herring sperm DNA at 300 V and 950 uF using the IBI GENEZAPPER (IBI). The transfected cells were incubated in CHO media [10% fetal calf serum, 100 U/ml penicillin-streptomycin, 2 mM glutamine, 1/500 hypoxanthine-thymidine (ATCC), 90% IMDM media (JRH BIOSCIENCES, Lenexa, Kans.), 0.7 mg/ml G418 (GIBCO)] in 5% CO₂ at 37° C. until colonies were visible. Each colony was separated and propagated. The cell clone with the highest number of human NK1R was selected for subsequent applications such as drug screening.

Assay Protocol using COS or CHO: The binding assay of human NK1R expressed in either COS or CHO cells is based on the use of ¹²⁵I-substance P (¹²⁵I-SP, from DU PONT, Boston, Mass.) as a radioactively labeled ligand which competes with unlabeled substance P or any other ligand for binding to the human NK1R. Monolayer cell cultures of COS or CHO were dissociated by the non-enzymatic solution (SPECIALTY MEDIA, Lavallette, N.J.) and resuspended in appropriate volume of the binding buffer (50 mM Tris pH 7.5, 5 mM MnCl₂, 150 mM NaCl, 0.04 mg/ml bacitracin, 0.004 mg/ml leupeptin, 0.2 mg/ml BSA, 0.01 mM phosphoramidon) such that 200 ul of the cell suspension would give rise to about 10,000 cpm of specific ¹²⁵I-SP binding (approximately 50,000 to 200,000 cells). In the binding assay, 200 ul of cells were added to a tube containing 20 ul of 1.5 to 2.5 nM of ¹²⁵I-SP and 20 ul of unlabeled substance P or any other test compound. The tubes were incubated at 4° C. or at room temperature for 1 hour with gentle shaking. The bound radioactivity was separated from unbound radioactivity by GF/C filter (BRANDEL, Gaithersburg, Md.) which was pre-wetted with 0.1% polyethylenimine. The filter was washed with 3 ml of wash buffer (50 mM Tris pH 7.5, 5 mM MnCl₂, 150 mM NaCl) three times and its radioactivity was determined by gamma counter. The activation of phospholipase C by NK1R may also be measured in CHO cells expressing the human NK1R by determining the accumulation of inositol monophosphate which is a degradation product of IP3. CHO cells are seeded in 12-well plate at 250,000 cells per well. After incubating in CHO media for 4 days, cells are loaded with 0.025 uCi/ml of ³H-myoinositol by overnight incubation. The extracellular radioactivity is removed by washing with phosphate buffered saline. LiCl is added to the well at final concentration of 0.1 mM with or without the test compound, and incubation is continued at 37° C. for 15 min. Substance P is added to the well at final concentration of 0.3 nM to activate the human NK1R. After 30 min of incubation at 37° C., the media is removed and 0.1 N HCl is added. Each well is sonicated at 4° C. and extracted with CHCl₃/methanol (1:1). The aqueous phase is applied to a 1 ml Dowex AG 1X8 ion exchange column. The column is washed with 0.1 N formic acid followed by 0.025 M ammonium formate-0.1 N formic acid. The inositol monophosphate is eluted with 0.2 M ammonium formate-0.1 N formic acid and quantitated by beta counter. In particular, the intrinsic tachykinin receptor antagonist activities of the compounds of the present invention may be demonstrated by these assays. The compounds of the following examples have activity in the aforementioned assays in the range of 0.05 nM to 10 μM. The activity of the present compounds may also be demonstrated by the assay disclosed by Lei, et al., British J. Pharmacol., 105, 261-262 (1992).

According to a further or alternative aspect, the present invention provides a compound of the present invention for use as a composition that may be administered to a subject in need of a reduction of the amount of tachykinin or substance P in their body.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives.

Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

The compositions containing compounds of the present invention may be presented in unit dosage form and may be prepared by any of the methods well known in the art of pharmacy. The term “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets or capsules for oral administration, single dose vials for injection, or suppositories for rectal administration. This list of unit dosage forms is not intended to be limiting in any way, but merely to represent typical examples in the pharmacy arts of unit dosage forms. The compositions containing compounds of the present invention may also be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient.

By “pharmaceutically acceptable” it is meant the carrier, diluent or excipient must be compatible with the other ingredients of the formulation and not deleterious to the recipient thereof.

The terms “administration of” or “administering a” compound should be understood to mean providing a compound of the invention to the individual in need of treatment in a form that can be introduced into that individuals body in a therapeutically useful form and therapeutically effective amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories. The term “therapeutically effective amount” refers to a sufficient quantity of the compounds of the present invention, in a suitable composition, and in a suitable dosage form to treat or prevent the noted disease conditions.

The compounds of the present invention may be administered in combination with another substance that has a complimentary effect to the tachykinin and substance P inhibitors of the present invention. Accordingly, in the prevention or treatment of emesis, a compound of the present invention may be used in conjunction with other anti-emetic agents, especially 5HT₃ receptor antagonists, such as ondensetron, granisetron, tropisetron, palonosetron and zatisetron, a corticosteroid, such as dexamethasone, or GABA_{B} receptor agonists, such as baclofen. Likewise, for the prevention or treatment of migraine a compound of the present invention may be used in conjunction with other anti-migraine agents, such as ergotamines or 5HT₁ agonists, especially sumatriptan, naratriptan, zolmatriptan or rizatriptan.

It will be appreciated that for the treatment of depression or anxiety, a compound of the present invention may be used in conjunction with other anti-depressant or anti-anxiety agents, such as norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), α-adrenoreceptor antagonists, atypical anti-depressants, benzodiazepines, 5-HT_{1A} agonists or antagonists, especially 5-HT_{1A} partial agonists, corticotropin releasing factor (CRF) antagonists, and pharmaceutically acceptable salts thereof. For the treatment or prevention of eating disorders, including obesity, bulimia nervosa and compulsive eating disorders, a compound of the present invention may be used in conjunction with other anorectic agents. It will be appreciated that for the treatment or prevention of pain or nociception or inflammatory diseases, a compound of the present invention may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent.

It will be appreciated that when using any combination described herein, both the compound of the present invention and the other active agent(s) will be administered to a patient, within a reasonable period of time. The compounds may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially. Therefore, by way of example, one active component may be administered as a tablet and then, within a reasonable period of time, the second active component may be administered either as an oral dosage form such as a tablet or a fast-dissolving oral dosage form. By a “fast dissolving oral formulation” is meant, an oral delivery form which when placed on the tongue of a patient, dissolves within about 10 seconds. By “reasonable period of time” is meant a time period that is not in excess of about 1 hour. That is, for example, if the first active component is provided as a tablet, then within one hour, the second active component should be administered, either in the same type of dosage form, or another dosage form which provides effective delivery of the medicament.

The compounds of this invention may be administered to patients (humans and animals, including companion animals, such as dogs, cats and horses) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician.

In the treatment of the conditions associated with an excess of tachykinins, a suitable dosage level of the compounds of the present invention, or pharmaceutically acceptable salts thereof, is about 0.001 to 50 mg/kg per day, in particular about 0.01 to about 25 mg/kg, such as from about 0.05 to about 10 mg/kg per day. The dosage range will generally be about 0.5 to 1000 mg per patient per day, which may be administered in single or multiple doses. Preferably, the dosage range will be about 0.5 mg to 500 mg per patient per day; more preferably about 0.5 mg to 200 mg per patient per day; and even more preferably about 5 mg to 50 mg per patient per day. Specific dosages of the compounds of the present invention, or pharmaceutically acceptable salts thereof, for administration include 1 mg, 5 mg, 10 mg, 30 mg, 100 mg, and 500 mg. Pharmaceutical compositions of the present invention may be provided in a formulation comprising about 0.5 mg to 1000 mg active ingredient; more preferably comprising about 0.5 mg to 500 mg active ingredient; or 0.5 mg to 250 mg active ingredient; or 1 mg to 100 mg active ingredient. Specific pharmaceutical compositions for treatment or prevention of excess tachykinins comprise about 1 mg, 5 mg, 10 mg, 30 mg, 100 mg, and 500 mg of active ingredient.

Several methods for preparing the compounds of this invention are illustrated in the following Examples. Starting materials and the requisite intermediates are in some cases commercially available, or can be prepared according to literature procedures or as illustrated herein. All NMR spectra were obtained on instrumentation at a field strength of 400 or 500 MHz in either CDCl₃ or CD₃OD with reported chemical shifts as δ. The HPLC/MS analyses were obtained using an Agilent 1100 Series HPLC in combination with a Waters Micromass ZQ mass spectrometer. The HPLC RP column was a Waters Exterra MS-C18 (5 μm) 3.0×50 mm column eluting with a 10-100% acetonitrile/water (both containing 0.05% TFA) gradient over 3.75 min with a run time of 5.50 min. UV monitoring was done at 210 nM. Retention times (Rt) are reported in minutes based on the MS data. The reported m/e value was usually the parent molecular ion, except when the 100% ion was not the parent ion as also indicated. Preparative chiral HPLC was done with the indicated Chiracel 25×250 mm columns eluting at 9 mL per min with the indicated percent isopropanol/heptanes solvent mixture. Retention times (Rt) are reported in minutes based on the UV chromatogram monitored at 210 or 254 nm.

### EXAMPLE 1

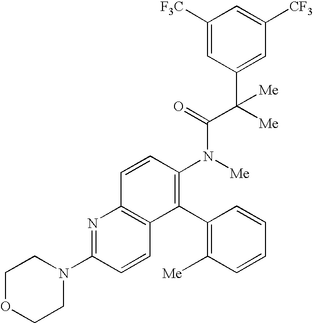

### 2-[3,5-bis(Trifluoromethyl)phenyl]-N,2-dimethyl-N-[5-(2-methylphenyl)-2-morpholin-4-ylquinolin-6-yl]propanamide

### Step A: 2-Morpholin-4-yl-6-nitroquinoline

To a solution of 3.93 g (18.8 mmol) of 2-chloro-6-nitroquinoline in MeOH in a pressure tube was added 2.0 g (23 mmol) morpholine and 7 mL TEA. The pressure tube was sealed and the reaction mixture was heated at 80° C. for 20 hr. The resulting mixture was cooled to RT and ˜20 mL of 5 N aq. NaOH was added. The resulting mixture was stirred at RT for 5 min. then diluted with CHCl₃. The layers were separated and the aqueous layer further extracted with CHCl₃. The combined organic extracts were dried over drying agent and filtered. The solvent was removed under vacuum to afford the title compound which was used without further purification. MS (MH)⁺: 260.1.

### Step B: 2-Morpholin-4-ylquinolin-6-amine

A solution of 2-morpholin-4-yl-6-nitroquinoline (step A) in MeOH was hydrogenated at 50 PSI hydrogen over 0.5 g of PtO₂ for 0.5 hr at RT. The catalyst was filtered through filter aid and the solvent of the filtrate was evaporated under vacuum to afford the title compound which was used without further purification. MS (MH)⁺: 230.2.

### Step C: 5-Bromo-2-morpholin-4-ylquinolin-6-amine

To a solution of 3.6 g (15.7 mmol) of 2-morpholin-4-ylquinolin-6-amine (step B) in CH₂Cl₂ at 0° C. was added a solution of 0.81 mL (15.7 mmol) bromine in CH₂Cl₂. The resulting suspension was stirred at 0° C. for 0.5 hr then was quenched by the addition of 2 N aq. NaOH. The layers were separated and the aqueous layer further extracted with CHCl₃. The combined organic extracts were dried over drying agent, filtered and the solvent was removed under vacuum. The residue was purified by column chromatography on silica gel eluting with hexanes/EtOAc (1/1) to afford the title compound. ¹H-NMR (CD₃OD): δ 8.18 (1H, d), 7.50 (1H, d), 7.20 (1H, d), 7.15 (1H, d), 3.82 (4H, m), 3.60 (4H, m). MS (MH)⁺: 310.1.

### Step D: Benzyl (5-bromo-2-morpholin-4-ylquinolin-6-yl)carbamate

To a solution of 5-bromo-2-morpholin-4-ylquinolin-6-amine (step C) in HOAc, under nitrogen atmosphere at 0° C. was added CBZ-Cl. The cooling bath was removed and the reaction mixture was stirred at RT for 16 hr. The resulting mixture was diluted with methylene chloride and quenched with excess water. The layers were separated and the organic layer dried over drying agent, filtered and the solvent evaporated under vacuum to afford the crude title compound which was used without further purification, and was contaminated with some starting material.

### Step E: Benzyl [5-(2-methylphenyl)-2-morpholin-4-ylquinolin-6-yl]carbamate

To a solution of 1.4 g of the intermediate of step D in toluene under nitrogen atmosphere was added 0.76 g (1.5 equiv) 2-methylphenylboronic acid, 1.2 mL EtOH, 3.6 mL sat. aq. NaHCO₃ and 0.15 g (cat.) tetrakis(triphenylphosphine)Pd(0). The reaction mixture was heated at reflux for 16 hr. The reaction mixture was cooled to RT, diluted with EtOAc and water then transferred to a separatory funnel. The organic layer was separated, washed with water, dried over anhydrous sodium sulfate, filtered and the solvent evaporated under vacuum. The residue was purified by column chromatography on silica gel eluting with hexanes/EtOAc (1/1) to afford the title compound. MS (MH)⁺: 454.2.

### Step F: 5-(2-Methylphenyl)-2-morpholin-4-ylquinolin-6-amine

A solution of benzyl [5-(2-methylphenyl)-2-morpholin-4-ylquinolin-6-yl]carbamate (step E) in MeOH was hydrogenated at 50 PSI hydrogen over a catalytic amount of 10% Pd—C for several hr at RT. The catalyst was filtered through filter aid and the solvent of the filtrate was evaporated under vacuum to afford the title compound which was used without further purification. ¹H-NMR (CD₃OD): δ 7.60 (1H, d), 7.45-7.05 (6H, m), 6.97 (1H, d), 3.82 (4H, m), 3.60 (4H, m), 1.98 (3H, s). MS (MH)⁺: 320.2.

### Step G: 2-[3,5-bis(Trifluoromethyl)phenyl]-2-methyl-N-[5-(2-methylphenyl)-2-morpholin-4-ylquinolin-6-yl]propanamide

To a solution of 0.06 g of 5-(2-methylphenyl)-2-morpholin-4-ylquinolin-6-amine (step F) in dry methylene chloride under nitrogen atmosphere at RT was added 0.04 g DMAP, 0.08 g EDC and 0.08 g of 2-[3,5-bis(trifluoromethyl)phenyl]-2-methylpropanoic acid. The resulting mixture was stirred at RT for 24 hr. The solvent evaporated under vacuum and the residue was purified by preparative TLC eluting with EtOAc/hexanes to afford the title compound. LC-MS (MH)⁺: 602.2.

### Step H: 2-[3,5-bis(Trifluoromethyl)phenyl]-N,2-dimethyl-N-[5-(2-methylphenyl)-2-morpholin-4-ylquinolin-6-yl]propanamide

To a solution of 0.60 g (1.24 mmol) of 2-[3,5-bis(trifluoromethyl)phenyl]-2-methyl-N-[5-(2-methylphenyl)-2-morpholin-4-ylquinolin-6-yl]propanamide (step G) in dry THF under nitrogen atmosphere at −78° C. was added by syringe a solution of KHMDS in toluene. The resulting mixture was stirred at −78° C. for 0.33 hr then treated with excess MeI. The cooling bath was removed and the resulting reaction mixture was stirred at ambient T for 1 hr. The reaction mixture was quenched with water then diluted with EtOAc. The organic layer was separated, dried over sodium sulfate, filtered and the solvent was evaporated under vacuum. The residue was purified by prep TLC eluting with EtOAc/hexanes to afford the title compound. MS (MH)⁺: 616.3.

### EXAMPLE 2

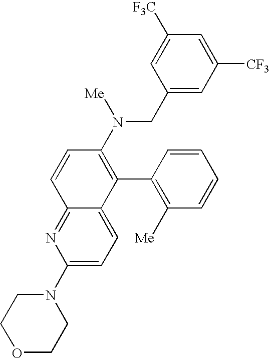

### N-[3,5-bis(Trifluoromethyl)benzyl]-N-methyl-5-(2-methylphenyl)-2-morpholin-4-ylquinolin-6-amine

### Step A: N-[3,5-bis(Trifluoromethyl)benzyl]-5-(2-methylphenyl)-2-morpholin-4-ylquinolin-6-amine

To a solution 0.02 g of 5-(2-methylphenyl)-2-morpholin-4-ylquinolin-6-amine (Example 1, step F) in dry methylene chloride under nitrogen atmosphere at RT was added 0.15 g of 3,5-bis(trifluoromethyl)benzaldehyde and 0.53 g NaBH(OAc)₃. The resulting mixture was stirred at RT for 16 hr. The solvent evaporated under vacuum and the residue was purified by prep TLC eluting with EtOAc/hexanes to afford the title compound. MS (MH)⁺: 546.3

### Step B: N-[3,5-bis(Trifluoromethyl)benzyl]-N-methyl-5-(2-methylphenyl)-2-morpholin-4-ylquinolin-6-amine

To a solution of N-[3,5-bis(trifluoromethyl)benzyl]-5-(2-methylphenyl)-2-morpholin-4-ylquinolin-6-amine (step A) in MeOH under nitrogen atmosphere at 0° C. was added 30%, aq. formaldehyde and sodium acetate. The resulting mixture was stirred at 0° C. for 0.5 hr and then NaBH₃CN was added. The reaction mixture was stirred at RT for 2 hr. The solvent evaporated under vacuum and the residue was purified by prep TLC eluting with EtOAc/hexanes to afford the title compound. MS (MH)⁺: 560.3

### EXAMPLE 3

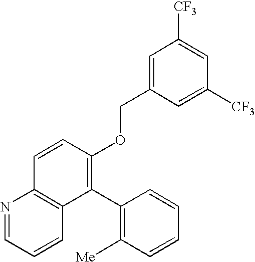

### 6-{[3,5-bis(Trifluoromethyl)benzyl]oxy}-5-(2-methylphenyl)quinoline

### Step A: 5-Bromoquinolin-6-amine

The title compound was prepared from 6-aminoquinoline according to the procedure of Example 1, step C. MS: (MH)⁺: 225.0

### Step B: tert-Butyl (5-bromoquinolin-6-yl)carbamate

To a solution of 5-bromoquinolin-6-amine (step A) in CH₂Cl₂ at RT was added ditert-butyl dicarbonate and a catalitical amount of DMAP. The reaction mixture was stirred at RT for several hours and worked up in the usual manner to afford the title compound ¹H-NMR (CDCl₃): δ 9.0 (1H, d), 8.65 (1H, d), 8.10 (1H, d), 7.60 (1H, d), 7.55 (1H, m), 1.40 (9H, s).

### Step C: tert-Butyl [5-(2-methylphenyl)quinolin-6-yl]carbamate

The title compound was prepared from tert-butyl (5-bromoquinolin-6-yl)carbamate (step B) and 2-methylphenylboronic acid according to the procedure of Example 1, step E.

### Step D: 5-(2-Methylphenyl)quinolin-6-amine trifluoroacetic acid salt

A solution of 0.23 g (0.69 mmol) of the intermediate of step C in 10 mL CH₂Cl₂/TFA (2/1) was stirred at RT 1 hr. The solvent evaporated under vacuum to afford the title compound which was used without further purification. MS (MH)⁺: 235.1.

### Step E: 5-(2-Methylphenyl)quinolin-6-ol

A solution of 5-(2-methylphenyl)quinolin-6-amine trifluoroacetic acid salt (step D) in 1.5 mL 3 N aq. H₂SO₄ at 0° C. was added an aq. solution NaNO₂. The reaction mixture was stirred at 0° C. for 0.33 hr at which time 10 mg urea was added. The reaction mixture was stirred at 0° C. for 10 min at which time 10 mL of 50% aq. H₂SO₄ was added. The reaction mixture was heated at 110° C. for 2 hr. The reaction mixture was cooled to RT then to 0° C. in an ice bath. The pH of the mixture was adjusted to pH˜3-4 by the careful addition of 5 N aq. NaOH then to pH˜9 by addition of sat. aq. NaHCO₃. The mixture was extracted with EtOAC (3×10 mL). The combined organic extracts were dried over drying agent, filtered and the solvent was removed under vacuum. The residue was purified by prep TLC to afford the title compound. MS (MH)⁺: 236.1

### Step F: 6-{[3,5-bis(Trifluoromethyl)benzyl]oxy}-5-(2-methylphenyl)quinoline

To a solution of 0.085 g of 5-(2-methylphenyl)quinolin-6-ol (step E) in acetone was added 0.2 g CsCO₃ and 0.2 mL of 3,5-bis(trifluoromethyl)benzyl bromide. The resulting mixture was heated at 50° C. for 16 hr. After cooling to RT, the solids were filtered and the solvent of the filtrate evaporated under vacuum. The residue was purified by prep TLC eluting to afford the title compound. MS (MH)⁺: 462.5

### EXAMPLE 4

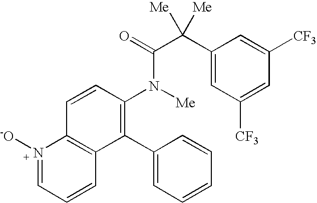

### 2-[3,5-bis(Trifluoromethyl)phenyl]-N,2-dimethyl-N-(5-phenylquinolin-6-yl)propanamide 1-oxide

### Step A: tert-Butyl (5-phenylquinolin-6-yl)carbamate

The title compound was prepared from tert-butyl (5-bromoquinolin-6-yl)carbamate (Example 3, step B) and phenylboronic acid according to the procedure of Example 1, step E. MS (MH)⁺: 325.1

### Step B: 5-Phenylquinolin-6-amine

The title compound was prepared from tert-butyl (5-phenylquinolin-6-yl)carbamate (step A) according to the procedure of Example 3, step D, and was converted into a free base by preparative TLC with a mixture solvent of 1/9 2 N NH₃ in MeOH/CHCl₃. ¹H-NMR (CDCl₃): δ 8.67 (1H, d), 8.00 (1H, m). 7.68 (1H, d), 7.59 (2H, m), 7.50 (1H, m), 7.20 (2H, d), 7.28 (2H, m), 7.20 (1H, m), 3.85 (2H, bs). MS (MH)⁺: 225.0.

### Step C: 2-[3,5-bis(Trifluoromethyl)phenyl]-2-methyl-N-(5-phenylquinolin-6-yl)propanamide

To a solution of 0.51 g of 5-phenylquinolin-6-amine trifluoroacetic acid salt (step B) in dry methylene chloride under nitrogen atmosphere at RT was added DIPEA and 2-[3,5-bis(trifluoromethyl)phenyl]-2-methylpropanoyl chloride. The resulting mixture was heated at 50° C. for 16 hr. The mixture was cooled to RT, quenched with 2 N. aq. NaOH and extracted with EtOAc. The combined organic extracts were dried over drying agent and filtered. The solvent evaporated under vacuum to afford the title compound. MS (MH)⁺: 503.2

### Step D: 2-[3,5-bis(Trifluoromethyl)phenyl]-N,2-dimethyl-N-(5 phenylquinolin-6-yl)propanamide

To a solution of 2-[3,5-bis(trifluoromethyl)phenyl]-2-methyl-N-(5-phenylquinolin-6-yl)propanamide (step C) in dry DMF under nitrogen atmosphere at RT was added NaH dispersion in oil. The resulting mixture was stirred at RT then treated with excess MeI at RT. The resulting reaction mixture was stirred at ambient T until complete. The reaction mixture was quenched with water then diluted with EtOAc. The organic layer was separated, dried over sodium sulfate, filtered and the solvent was evaporated under vacuum. The residue was purified by prep TLC eluting with EtOAc/hexanes to afford the title compound. MS (MH)⁺: 517.2.

### Step E: 2-[3,5-bis(Trifluoromethyl)phenyl]-N,2-dimethyl-N-(5-phenylquinolin-6-yl)propanamide 1-oxide

To a solution of 0.54 g of 2-[3,5-bis(trifluoromethyl)phenyl]-N,2-dimethyl-N-(5-phenylquinolin-6-yl)propanamide (step D) in CHCl₃ was added 1.0 g (4 equiv.) of 75% m-CPBA. The resulting mixture was stirred at RT for 16 hr then 2 N aq. NaOH was added. The resulting mixture was stirred at RT for 0.5 hr then was extracted with methylene chloride. The combined extracts were washed with brine, dried over drying agent, filtered and the solvent was evaporated under vacuum to afford the title compound. MS (MH)⁺: 533.2.

### EXAMPLE 5

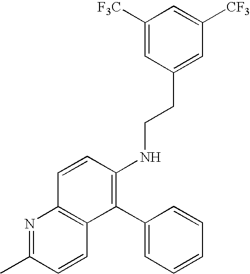

### N-{2-[3,5-bis(Trifluoromethyl)phenyl]ethyl}-2-methyl-5-phenylquinolin-6-amine

### Step A: 2-Methylquinolin-6-amine

The title compound was prepared from 2-methyl-6-nitroquinoline according to the procedure of Example 1, step B. MS (MH)⁺: 159.1.

### Step B: 5-Bromo-2-methylquinolin-6-amine

The title compound was prepared from 2-methylquinolin-6-amine (step A) according to the procedure of Example 1, step C. MS (MH)⁺: 238.0.

### Step C: tert-Butyl (5-bromo-2-methylquinolin-6-yl)carbamate

The title compound was prepared from 5-bromo-2-methylquinolin-6-amine (step B) and ditert-butyl dicarbonate according to the procedure of Example 3, step B.

### Step D: tert-Butyl (2-methyl-5-phenylquinolin-6-yl)carbamate

The title compound was prepared from tert-butyl (5-bromo-2-methylquinolin-6-yl)carbamate (step C) and phenylboronic acid according to the procedure of Example 1, step E. MS\ (MH)⁺: 335.3.

### Step E: 2-Methyl-5-phenylquinolin-6-amine

The title compound was prepared from tert-butyl (2-methyl-5-phenylquinolin-6-yl)carbamate (step D) according to the procedure of Example 3, step D, and was converted into a free base by preparative TLC with a mixture solvent of 1/9 2 N NH₃ in MeOH/CHCl₃. ¹H-NMR (CDCl₃): δ 7.87 (1H, d), 7.60−7.55 (2H, m), 7.50 (1H, m), 7.35 (2H, d), 7.15 (1H, d), 7.10 (1H, d), 3.80 (2H, bs), 2.70 (3H, s). MS (MH)⁺: 235.1.

### Step F: 2-[3,5-bis(Trifluoromethyl)phenyl]-N-(2-methyl-5-phenylquinolin-6-yl)acetamide

The title compound was prepared from 2-methyl-5-phenylquinolin-6-amine trifluoroacetic acid salt (step E) and of 2-[3,5-bis(trifluoromethyl)phenyl]-2-methylpropanoic acid according to the procedure of Example 1, step G. MS (MH)⁺: 489.3.

### Step G: N-{2-[3,5-bis(Trifluoromethyl)phenyl]ethyl}-2-methyl-5-phenylquinolin-6-amine

To a solution of 0.60 g (1.24 mmol) of 2-[3,5-bis(trifluoromethyl)phenyl]-N-(2-methyl-5-phenylquinolin-6-yl)acetamide (step F) in dry THF under nitrogen atmosphere was added by syringe a solution of borane-THF complex in THF. The resulting mixture was stirred at RT for 16 hr. The reaction mixture was quenched with MeOH and the solvent was evaporated under vacuum. The residue was purified by prep TLC to afford the title compound. MS (MH)⁺: 475.1.

### EXAMPLE 6

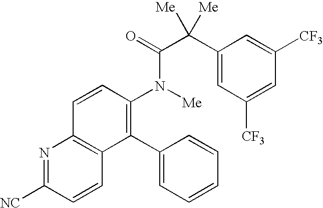

### 2-[3,5-bis(Trifluoromethyl)phenyl]-N-(2-cyano-5-phenylquinolin-6-yl)-N,2-dimethylpropanamide

### Step A: 2-[3,5-bis(Trifluoromethyl)phenyl]-N-(2-cyano-5-phenylquinolin-6-yl)-N,2-dimethylpropanamide

A solution of 0.25 g (0.47 mmol) 2-[3,5-bis(trifluoromethyl)phenyl]-N,2-dimethyl-N-(5-phenylquinolin-6-yl)propanamide 1-oxide (Example 4) in DMF was treated with 0.07 g (3 equiv.) NaCN, 0.4 mL (4 equiv.) TEA and 0.3 mL (5 equiv.) of trimethylsilyl chloride. The reaction mixture was stirred at room temperature for 72 hr and worked up in the usual manner to afford the title compound. MS (MH)⁺: 543.1.

### EXAMPLE 7

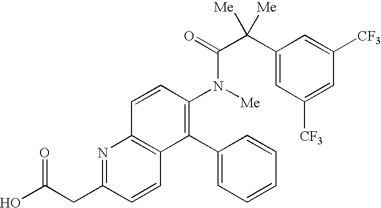

### {6-[{2-[3,5-bis(Trifluoromethyl)phenyl]-2-methylpropanoyl}(methyl)amino]-5-phenylquinolin-2-yl}acetic acid trifluoroacetic acid salt

### Step A: Ethyl {6-[{2-[3,5-bis(trifluoromethyl)phenyl]-2-methylpropanoyl}(methyl)amino]-5-phenylquinolin-2-yl}acetate

The title compound was prepared from 2-[3,5-bis(trifluoromethyl)phenyl]-N,2-dimethyl-N-(5-phenylquinolin-6-yl)propanamide 1-oxide (Example 4) and ethyl acetylacetate (ethyl 3-oxobutanoate) according to the procedure of Iwao and Kuraishi (J. Heterocycl. Chem. 1978, 15, 1425. 2-[3,5-Bis(trifluoromethyl)phenyl]-N,2-dimethyl-N-(5-phenylquinolin-6-yl)propanamide 1-oxide (0.17 g) and ethyl acetylacetate (ethyl 3-oxobutanoate, 0.062 g) were heated at 40° C. for 16 hr. The mixture was poured onto ice water. At room temperature, the solid was collected by filtration and stirred with 10 mL 10% aq. HCl for 0.5 hr. The mixture was made basic (pH=˜12), extracted with CHCl₃. The combined extracts were dried over MgSO₄. The solvent was removed under vacuum and the crude material was purified by preparative TLC. MS (MH)⁺: 603.1.

### Step B: {6-[{2-[3,5-bis(Trifluoromethyl)phenyl]-2-methylpropanoyl}(methyl)amino]-5-phenylquinolin-2-yl}acetic acid trifluoroacetic acid salt

To a solution of 0.015 g of ethyl {6-[{2-[3,5-bis(trifluoromethyl)phenyl]-2-methylpropanoyl}(methyl)amino]-5-phenylquinolin-2-yl}acetate (step A) in CHCl₃ was added 0.32 g of 75% m-CPBA. The resulting mixture was stirred at RT for 16 hr then 1 N aq. NaOH was added. The resulting mixture was stirred at RT for 0.5 hr then was extracted with CHCl₃. The combined extracts were dried over drying agent, filtered and the solvent was evaporated under vacuum. The residue was treated with 2 N aq. NaOH in MeOH for 5 hr. The reaction mixture was acidified to pH 4-7 with 2 N aq. HCl then was extracted with CHCl₃. The combined extracts were dried over drying agent, filtered and the solvent was evaporated under vacuum. The residue was purified by reverse phase HPLC to afford the title compound. MS (MH)⁺: 575.1.

### EXAMPLE 8

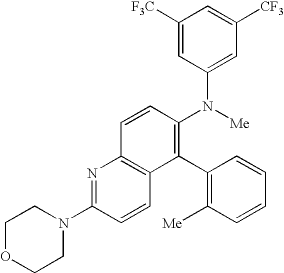

### N-[3,5-bis(Trifluoromethyl)phenyl]-N-methyl-4-(2-methylphenyl)-2-morpholin-4-ylquinolin-6-amine

### Step A: 6-nitroquinoline-2,4-diol

To a mixture of 3 g of quinoline-2,4-diol in 27 mL conc. H₂SO₄ at RT was added 0.7 mL fuming HNO₃. The reaction mixture was stirred at RT for 0.5 hr. The reaction mixture was poured carefully onto ice. The solids were filtered and washed with water, EtOAc and dried overnight to afford 3.5 g of the title compound which was used without further purification.

### Step B: 2,4-Dichloro-6-nitroquinoline

A mixture of 3 g of 6-nitroquinoline-2,4-diol in 40 mL POCl₃ was heated at 80° C. for 2 hr. The reaction mixture was cooled to RT and the volatiles removed under vacuum. The residue was carefully quenched with ice/water. The solids were filtered and washed with water, dried to afford 3.2 g of the title compound which was used without further purification.

### Step C: 4-Chloro-2-morpholin-4-yl-6-nitroquinoline

The title compound was prepared from 2,4-dichloro-6-nitroquinoline (step B) and morpholine according to the procedure of Example 1, step A. ¹H-NMR (CDCl₃): δ 9.00 (1H, s), 8.40 (1H, d), 7.73 (1H, d), 7.20 (1H, s), 3.90 (4H, m), 3.85 (4H, m). MS:=(MH)⁺: 294.1

### Step D: 4-(2-Methylphenyl)-2-morpholin-4-yl-6-nitroquinoline

The title compound was prepared from 4-chloro-2-morpholin-4-yl-6-nitroquinoline (step C) and 2-methylphenylboronic acid according to the procedure of Example 1, step E, and was used directly in the next step.

### Step E: 4-(2-Methylphenyl)-2-morpholin-4-ylquinolin-6-amine

The title compound was prepared from 4-(2-methylphenyl)-2-morpholin-4-yl-6-nitroquinoline (step D) according to the procedure of Example 1, step B. ¹H-NMR (CD₃OD): δ 7.60 (1 H, d), 3.35 (2H, m), 7.30 (1H, m), 7.20 (1H, d), 7.11 (1H, d), 6.92 (2H, s), 6.47 (1H, s), 3.82 (4H, m), 3.61 (4H, m), 2.07 (3H, s). MS (MH)⁺: 320.4.

### Step F: tert-Butyl [4-(2-methylphenyl)-2-morpholin-4-ylquinolin-6-yl]carbamate

The title compound was prepared from 4-(2-methylphenyl)-2-morpholin-4-ylquinolin-6-amine (step E) according to the procedure of Example 3, step B.

### Step G: N-Methyl-4-(2-methylphenyl)-2-morpholin-4-ylquinolin-6-amine

To a solution of tert-butyl [4-(2-methylphenyl)-2-morpholin-4-ylquinolin-6-yl]carbamate (step F) in dry THF under nitrogen atmosphere at 0° C. was added by syringe a solution of LiAlH₄ in THF. The resulting mixture was warmed to RT then heated at 80° C. for 16 hr. The reaction mixture was quenched in the normal manner (water and aq. NaOH base quench), solids filtered and the solvent was evaporated under vacuum to afford the title compound. MS (MH)⁺: 334.3.

### Step H: N-[3,5-bis(Trifluoromethyl)phenyl]-N-methyl-4-(2-methylphenyl)-2-morpholin-4-ylquinolin-6-amine

To a solution of 74 mg of 1-bromo-3,5-bis(trifluoromethyl)benzene in dry THF in a pressure tube under nitrogen atmosphere was added 31 mg of Na-Ot-Bu, 9 mg of Pd(Cl)₂DPPF and 18 mg of DPPG ligand. To the resulting solution was added 71 mg of N-methyl-4-(2-methylphenyl)-2-morpholin-4-ylquinolin-6-amine (step G). The pressure tube was purged with nitrogen, sealed and the reaction mixture was heated at 100° C. for 4 hr. The resulting mixture was cooled to RT and the solvent was removed under vacuum. The residue was purified by prep TLC eluting with EtOAc/hexanes to afford the title compound. ¹H-NMR (CD₃OD): δ 7.86 (1H, d), 7.45 (1H, d), 7.35-7.25 (3H, m), 7.20 (1H, d), 7.15 (1H, s), 7.10 (2H, s), 3.87 (4H, m), 3.75 (4H, m), 2.05 (3H, s). MS (MH)⁺: 546.4.

### Table 1

The compounds in Table 1 were synthesized using the foregoing methodology, but substituting the appropriately substituted reagent as described in the foregoing examples. The requisite starting materials were commercially available, described in the literature or readily synthesized by one skilled in the art of organic synthesis without undue experimentation.

| | | parent ion |
|---|---|---|
| Ex. # | Compound | (MH⁺) m/z |
| 9 | 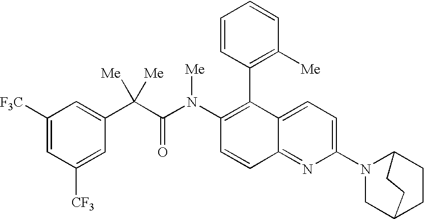 | 640.4 |
| 10 | 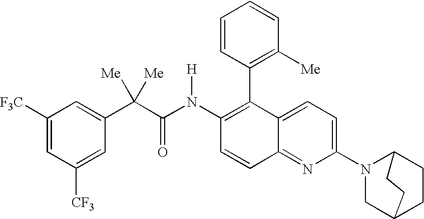 | 626.3 |
| 11 | 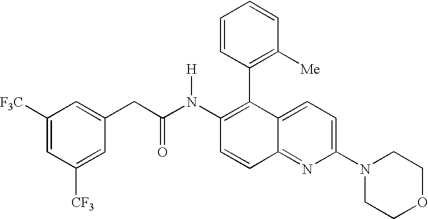 | 574.2 |
| 12 | 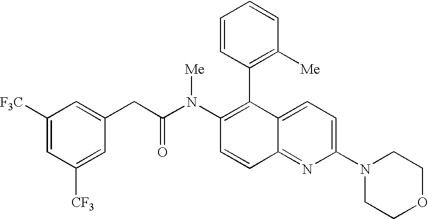 | 588.2 |
| 13 | 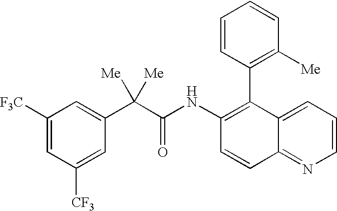 | 517.2 |
| 14 | 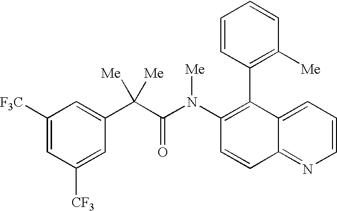 | 531.2 |
| 15 | 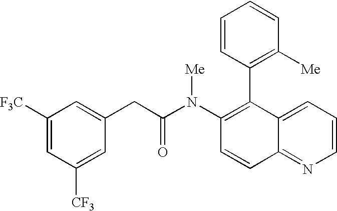 | 503.2 |
| 16 | 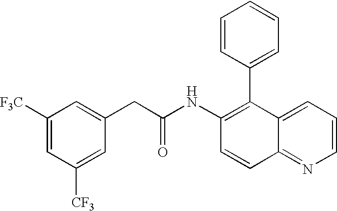 | 475.4 |
| 17 | 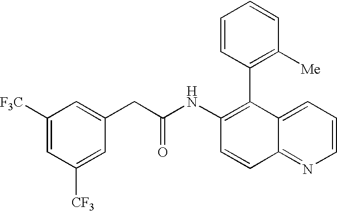 | 489.2 |
| 18 | 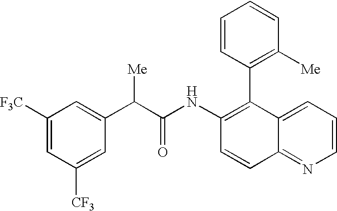 | 503.1 |
| 19 | 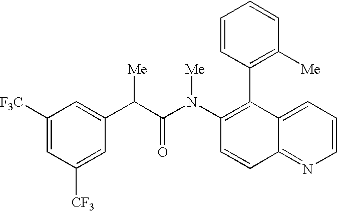 | 517.2 |
| 20 | 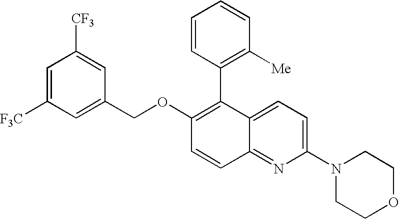 | 547.2 |
| 21 | 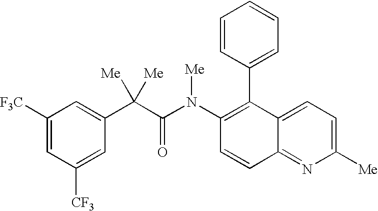 | 531.3 |
| 22 | 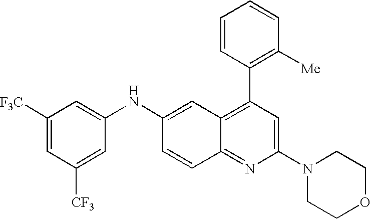 | 532.3 |
| 23 | 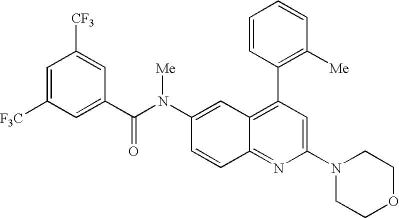 | 574.3 |
| 24 | 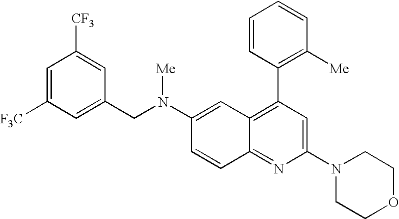 | 560.3 |
| 25 | 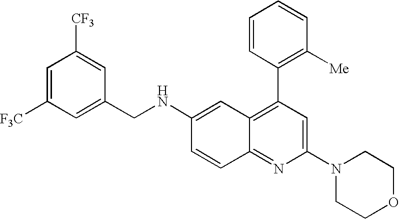 | 546.4 |
| 26 | 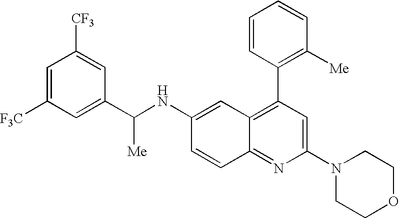 | 560.5 |
| 27 | 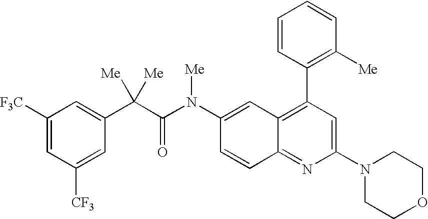 | 616.4 |
| 28 | 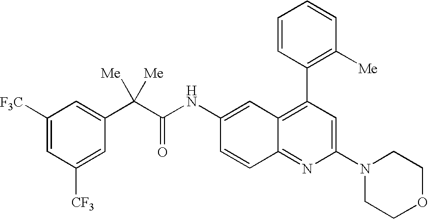 | 602.3 |
| 29 | 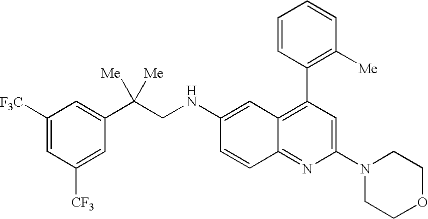 | 588.3 |
| 30 | 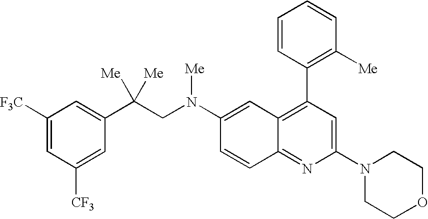 | 602.7 |
| 31 | 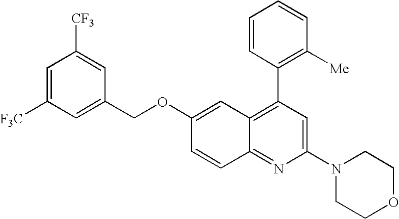 | 547.1 |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

What is claimed is:

1. A compound of the formula I: 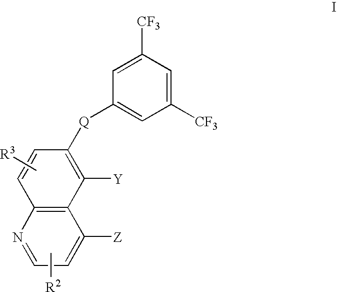
and N-oxides thereof,
wherein:
Q is selected from the group consisting of:
(1) —O—CH₂—,
(2) —O—CH(CH₃)—,
(3) —O—CH(CH₂OH)—,
(4) —N(R⁵)—C(R⁶R⁷)—,
wherein R⁵, R⁶ and R⁷ are independently selected from:
(a) hydrogen, and
(b) —CH₃,
(5) —N(R⁵)—,
(6) —N(R⁵)—CO—C(R⁶R⁷)—, and
(7) —N(R⁵)—CH₂—C(R⁶R⁷)—;
Y and Z are selected from hydrogen and phenyl, wherein one of Y and Z is hydrogen and the other of Y and Z is phenyl, and wherein the phenyl is substituted with R¹², R¹³ and R¹⁴, where R¹², R¹³ and R¹⁴ are independently selected from:
(1) hydrogen,
(2) halo, and
(3) C₁₋₆ alkyl;
R² and R³ are independently selected from the group consisting of:
(1) hydrogen,
(2) C₁₋₆ alkyl, which is unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) halo,
(g) —NR⁹R¹⁰, wherein R⁹ and R¹⁰ are independently selected from:
(I) hydrogen,
(II) C₁₋₆ alkyl,
(III) phenyl,
(IV) (C₁₋₆ alkyl)-phenyl,
(V) (C₁₋₆ alkyl)-hydroxy, and
(VI) (C₁₋₆ alkyl)-(C₁₋₄ alkoxy),
or where —NR⁹R¹⁰ forms a morpholine, piperidine or quinuclidine ring
(h) —NR⁹—COR¹¹, wherein R¹¹ is independently selected from:
(I) hydrogen,
(II) C₁₋₆ alkyl,
(III) phenyl,
(IV) (C₁₋₆ alkyl)-phenyl,
(V) (C₁₋₆ alkyl)-hydroxy, and
(VI) (C₁₋₆ alkyl)-(C₁₋₄ alkoxy),
(j) —NR⁹—CO₂R¹¹,
(k) —CO—NR⁹R¹⁰,
(l) —COR¹¹,
(m) —CO₂R¹¹,
(3) hydroxy,
(4) C₁₋₆alkoxy,
(5) oxo,
(6) halo,
(7) —CN,
(8) —CF₃,
(9) —NR⁹R¹⁰,
(10) —NR⁹—COR¹¹,
(11) —NR⁹—CO₂R¹¹,
(12) —CO—NR⁹—COR¹¹,
(13) —COR¹¹,
(14) —O—(CO)R¹¹,
(15) —CO₂R¹¹,
(16) -imidazolyl, and
(17) -triazolyl;
and pharmaceutically acceptable salts thereof and individual enantiomers and diastereomers thereof.

2. The compound of claim 1 of the formula Ia: 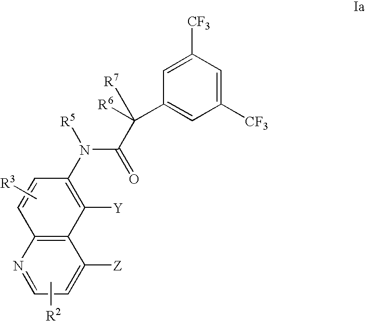
and N-oxides thereof, and pharmaceutically acceptable salts thereof and individual enantiomers and diastereomers thereof.

3. The compound of claim 1 of the formula Ib: 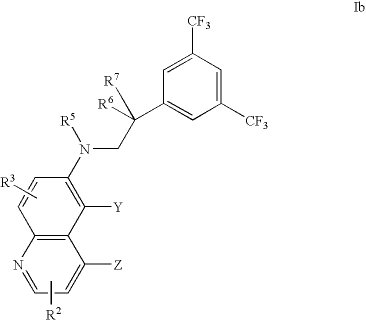
and N-oxides thereof, and pharmaceutically acceptable salts thereof and individual enantiomers and diastereomers thereof.

4. The compound of claim 1 of the formula Ic: 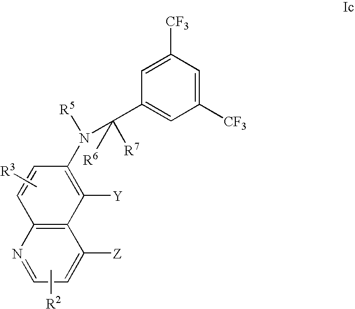
and N-oxides thereof, and pharmaceutically acceptable salts thereof and individual enantiomers and diastereomers thereof.

5. The compound of claim 1 of the formula Id: 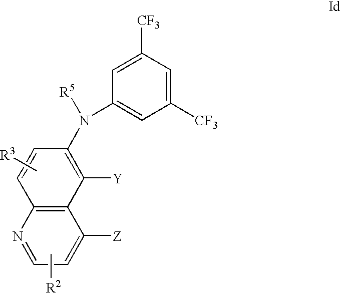
and N-oxides thereof, and pharmaceutically acceptable salts thereof and individual enantiomers and diastereomers thereof.

6. The compound of claim 1 of the formula Ie: 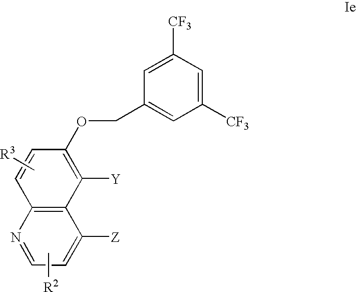
and N-oxides thereof, and pharmaceutically acceptable salts thereof and individual enantiomers and diastereomers thereof.

7. The compound of claim 1 wherein Y is phenyl and Z is hydrogen.

8. The compound of claim 1 wherein Y is hydrogen and Z is phenyl.

9. The compound of claim 1 wherein Y is 2-methyl-phenyl and Z is hydrogen.

10. The compound of claim 1 wherein Y is hydrogen and Z is 2-methyl-phenyl.

11. The compound of claim 1 wherein R² is selected from the group consisting of:
(1) hydrogen,
(2) morpholinyl,
(3) quinuclidinyl,
(4) C₁₋₆ alkyl, which is unsubstituted or substituted with one or more of the substituents selected from:
(a) morpholinyl,
(b) —CO₂(C₁₋₆ alkyl), and
(c) —CO₂H,
(5) hydroxy,
(6) —CO₂H, and
(7) —CN.

12. The compound of claim 1 wherein R² is hydrogen.

13. The compound of claim 1 wherein R² is morpholinyl.

14. The compound of claim 1 wherein R² is hydrogen.

15. The compound of claim 1 wherein R³ is hydrogen.

16. The compound of claim 1 wherein the compound is present as an N-oxide on the quinuclidinyl ring.

17. A compound which is selected from the group consisting of:
2-[3,5-bis(Trifluoromethyl)phenyl]-N,2-dimethyl-N-[5-(2-methylphenyl)-2-morpholin-4-ylquinolin-6-yl]propanamide;
N-[3,5-bis(Trifluoromethyl)benzyl]-N-methyl-5-(2-methylphenyl)-2-morpholin-4-ylquinolin-6-amine;
6-{[3,5-bis(Trifluoromethyl)benzyl]oxy}-5-(2-methylphenyl)quinoline;
2-[3,5-bis(Trifluoromethyl)phenyl]-N,2-dimethyl-N-(5-phenylquinolin-6-yl)propanamide 1-oxide;
N-{2-[3,5-bis(Trifluoromethyl)phenyl]ethyl}-2-methyl-5-phenylquinolin-6-amine;
2-[3,5-bis(Trifluoromethyl)phenyl]-N-(2-cyano-5-phenylquinolin-6-yl)-N,2-dimethylpropanamide;
{6-[{2-[3,5-bis(Trifluoromethyl)phenyl]-2-methylpropanoyl}(methyl)amino]-5-phenylquinolin-2-yl}acetic acid;
N-[3,5-bis(Trifluoromethyl)phenyl]-N-methyl-4-(2-methylphenyl)-2-morpholin-4-ylquinolin-6-amine;
and pharmaceutically acceptable salts thereof.

18. A pharmaceutical composition which comprises an inert carrier and a compound of claim 1 or a pharmaceutically acceptable salt thereof.

19. A method for the manufacture of a medicament for antagonizing the effect of substance P at its receptor site or for the blockade of neurokinin-1 receptors in a mammal comprising combining a compound of the present invention or a pharmaceutically acceptable salt thereof with a pharmaceutical carrier or diluent.

20. A method for the manufacture of a medicament for the treatment of a physiological disorder associated with an excess of tachykinins in a mammal comprising combining a compound of the present invention or a pharmaceutically acceptable salt thereof with a pharmaceutical carrier or diluent.
